(19) United States
(12) Patent Application Publication
(10) Pub. No.: US 2005/0021075 A1
(43) Pub. Date: Jan. 27, 2005

(54) Guidewire having deployable sheathless protective filter

(76) Inventors: Michael J. Bonnette, Minneapolis, MN (US); Eric J. Thor, Arden Hills, MN (US); John C. Riles, Minneapolis, MN (US)

Correspondence Address: HUGH D JAEGER, 1000 SUPERIOR BLVD, SUITE 302, WAYZATA, MN, 553911873, US

(21) Appl. No.: 10/748,451

(22) Filed: Dec. 30, 2003

Related U.S. Application Data
(60) Provisional application No. 60/437,166, filed on Dec. 30, 2002.

(51) Int. Cl. A61M 29/00

(52) U.S. Cl. 606/200

(57) ABSTRACT

A protective system or apparatus for use in vascular procedures includes a tubular guidewire, a control cable slidable within the tubular guidewire, and a sheathless filter. The control cable is attached to a distal end of the sheathless filter and the tubular guidewire is attached to a proximal end of the sheathless filter. Selective displacement of the control cable radially expands the sheathless filter to create a proximal exterior convex primary filter surface that is positionable downstream from a site of a vascular procedure. The sheathless filter also presents a distal interior concave secondary filter surface. Preferably, the sheathless filter is constructed of a braided wire framework in the form of a tube over which woven polymer fibers or strands are applied to create a filter mesh having a softer filter surface.

### RELATED APPLICATION

This application claims the benefit of U.S. Provisional Application No. 60/437,166, filed Dec. 30, 2002, incorporated herein in its entirety by reference.

### FIELD OF THE INVENTION

The present invention relates generally to the field of vascular medical devices. More specifically, the present invention relates to a protective system or apparatus for use in vascular procedures that includes a deployable filter that is sheathless.

### BACKGROUND OF THE INVENTION

Arterial disease involves damage that happens to the arteries in the body. Diseased arteries can become plugged with thrombus, plaque, or grumous material that may ultimately lead to a condition known as ischemia. Ischemia refers to a substantial reduction or loss of blood flow to the heart muscle or any other tissue that is being supplied by the artery and can lead to permanent damage of the affected region. While arterial disease is most commonly associated with the formation of hard plaque and coronary artery disease in the heart, similar damage can happen to many other vessels in the body, such as the peripheral vessels and cerebral vessels, due to the buildup of hard plaque or softer thrombus or grumous material within the lumen of an artery or vein.

A variety of vascular medical devices and procedures have been developed to treat diseased vessels. The current standard procedures include bypass surgery (where a new blood vessel is grafted around a narrowed or blocked artery) and several different types of non-surgical interventional vascular medical procedures, including angioplasty (where a balloon on a catheter is inflated inside a narrowed or blocked portion of an artery in an attempt to push back plaque or thrombotic material), stenting (where a metal mesh tube is expanded against a narrowed or blocked portion of an artery to hold back plaque or thrombotic material), and debulking techniques in the form of atherectomy (where some type of high speed or high power mechanism is used to dislodge hardened plaque) or thrombectomy (where some type of mechanism or infused fluid is used to dislodge grumous or thrombotic material). In each of these interventional vascular medical procedures, a very flexible guidewire is routed through the patient's vascular system to a desired treatment location and then a catheter that includes a device on the distal end appropriate for the given procedure is tracked along the guidewire to the treatment location.

Although interventional vascular procedures avoid many of the complications involved in surgery, there is a possibility of complications if some of the plaque, thrombus or other material breaks free and flows downstream in the artery or other vessel, potentially causing a stroke, a myocardial infarction (heart attack), or other tissue death. One solution to this potential complication is to use some kind of occlusive device or filtering device to block or screen the blood flowing downstream of the treatment location.

The use of a protective device in the form of an occlusive device or filtering device as part of a vascular procedure is becoming more common in debulking procedures performed on heart bypass vessels. Most heart bypass vessels are harvested and transplanted from the saphenous vein located along the inside of the patient's leg. The saphenous vein is a long, straight vein that has a capacity more than adequate to support the blood flow needs of the heart. Once transplanted, the saphenous vein is subject to a buildup of plaque or thrombotic materials in the grafted arterial lumen. Unfortunately, the standard interventional vascular treatments for debulking are only moderately successful when employed to treat saphenous vein coronary bypass grafts. The complication rate for a standard balloon angioplasty procedure in a saphenous vein coronary bypass graft is higher than in a native vessel with the complications including embolization, “no-reflow” phenomena, and procedural related myocardial infarction. Atherectomy methods including directional, rotational, and laser devices are also associated with a high degree of embolization resulting in a greater likelihood of infarction. The use of stents for saphenous vein coronary bypass grafts has produced mixed results. Stents provide for less restenosis, but they do not eliminate the risk of embolization and infarction incurred by standard balloon angioplasty.

In order to overcome the shortcomings of these standard non-surgical interventional treatments in treating saphenous vein coronary bypass graft occlusion, embolic protection methods utilizing a protective device distal to the lesion have been developed. The protective device is typically a filter or a balloon. Use of a protective device in conjunction with an atherectomy or thrombectomy device is intended to prevent emboli from migrating beyond the protective device and to allow the embolic particles to be removed, thereby subsequently reducing the risk of myocardial infarction. When the protective device is a balloon, the balloon is inserted and inflated at a point distal to the treatment site or lesion site. Therapy is then performed at the site and the balloon acts to block all blood flow, which prevents emboli from traveling beyond the balloon. Following treatment, some form of particle removal device must be used to remove the dislodged emboli prior to balloon deflation. U.S. Pat. No. 5,843,022 uses a balloon to occlude the vessel distal to a lesion or blockage site. The occlusion is treated with a high pressure water jet, and the fluid and entrained emboli are subsequently removed via an extraction tube. U.S. Pat. No. 6,135,991 describes the use of a balloon to occlude the vessel allowing blood flow and pressure to prevent the migration of emboli proximally from the treatment device. While effective as a protective device, balloons may result in damaged tissue due to lack of blood flow downstream of the treatment area due to the time required to inflate and deflate the balloon.

To overcome this disadvantage, most development in relation to occlusive devices has focused on devices that screen the blood through a filter arrangement. An early arterial filtering system utilizing a balloon catheter with a strainer device is described in U.S. Pat. No. 4,873,978. The device is inserted into a vessel downstream of the treatment site. The strainer responds to actuation of a separately introduced control cable to open and close a plurality of tines capable of retaining dislodged particles. After treatment, the strainer is collapsed and the entrapped emboli are removed from the body. The additional wire, however, creates additional complexity for the user.

More recently, filter designs have been deployed through the use of a single guidewire in which the filter device is transported to the deployment area within a sheath or catheter. Typical filters have either an umbrella shape to capture emboli or a tube shape in which the proximal end contains larger openings than the distal end so as to allow the blood and debris to enter the filter. The filter thus presents an operational face to the flow of blood within the vessel as provided by the distal end of the tubular filter that is concave in orientation. Particles are captured within the concave face of the filter and are then retracted out of the vessel when the entire device is removed from the body.

One of the challenges regarding filters is the manner in which a filter is transported to and from the area of interest. U.S. Pat. Nos. 6,042,598, 6,361,546, 6,371,970, 6,371,971 and 6,383,206 describe various examples of filter arrangements that are to be deployed through a sheath, while U.S. Pat. Nos. 6,080,170, 6,171,328, 6,203,561, 6,364,895, and 6,325,815 describe filters that are deployed by a catheter. For example, U.S. Pat. No. 6,371,971 describes a blood filter positioned by way of a single guidewire, covered by a sheath for advancement through a vessel. The sheath compresses struts of the filter while in transit. An interventional procedure requires deployment of the sheath along a guidewire downstream of the vascular occlusion. The sheath is retracted and the filter expands to a predetermined size. The filter is retrieved after the procedure by deploying the sheath back down the guidewire, capturing the filter and removing the system from the patient.

The disadvantage associated with this type of filter is the added thickness of the device due to the use of a sheath to deploy the filter. Typical sheath diameters exceed 0.040 inch. Insertion of the sheath can damage the vessel during routing and deployment to the occluded area and during removal. Moreover, the bulky sheath protecting the filter can hamper the debris removal or cause further embolization.

There is a need then for a protective device capable of embolization protection for vascular and arterial procedures without the design limitations of the existing approaches. Occlusive balloons can remain in place too long, thus increasing the risk of vessel damage downstream of the occlusion. Protective filters avoid this problem but suffer from complicated deployment and retraction schemes. Moreover, existing filters are limited in range due to the filter framework, which also may result in vessel damage. It would be desirable to provide an occlusive filter device that is easily deployable along a single guidewire without a large diameter sheath and that reduces the potential for vessel damage.

### SUMMARY OF THE INVENTION

The present invention is a protective system or apparatus for use in vascular procedures comprising a tubular guidewire; a control cable disposed within the lumen of the tubular guidewire; and a sheathless filter distally coupled to the control cable and proximally coupled to the tubular guidewire. The sheathless filter radially expands as the distal end of the sheathless filter is drawn toward the proximal end of the sheathless filter in response to displacement of the control cable relative to the tubular guidewire. The primary filter action is provided by the proximal outer convex surface of the sheathless filter, which is the first surface to come in contact with the flow of blood within a blood vessel.

In a preferred embodiment, the sheathless filter is comprised of a braided nitinol wire framework in the form of a tube to which woven strands are applied to create a filter mesh. In one embodiment, the woven strands are multifilament polymer fibers. In an alternate embodiment, the woven strands are nitinol wires of different diameters. The distal end of the control cable is attached to the distal end of the sheathless filter and the proximal end of the control cable extends beyond the proximal end of the tubular guidewire for access. Pulling the proximal end of the control cable draws the distal end of the sheathless filter toward the proximal end of the sheathless filter, which is attached to the tubular guidewire. The sheathless filter expands radially until it either fills the blood vessel or reaches a maximum expansion point at which filter mesh openings are still smaller than the smallest expected particle size of clinical significance. The sheathless filter may be locked in place to prevent premature closure of the sheathless filter. In one embodiment, the sheathless filter is provided with a radiopaque marker that provides an indication of the position and deployment state of the sheathless filter under fluoroscopy.

Unlike existing filters that have a concave operational surface, the proximal exterior surface of the deployed sheathless filter of the present invention has a convex shape that provides a first or primary filter surface. Particle removal from the convex filter surface is preferably accomplished in conjunction with a catheter-based aspiration device, such as a thrombectomy device, U.S. Pat. No. 5,370,609, commonly referred to as an AngioJet®. The use of an aspiration device enables removal of the majority of particles trapped by the convex filter surface prior to retraction of the sheathless filter through an evacuation lumen. Should debris escape the mesh of the proximal convex primary filter surface, the interior distal surface of the sheathless filter creates a concave secondary filter surface which also traps debris. Extraction requires reducing the sheathless filter diameter by retracting the control cable. The collapsed sheathless filter holds debris trapped by the secondary filter surface during the removal process.

In a preferred embodiment, the tubular guidewire is advanced over the control cable in a slidable fashion. A short tube, disposed intermediate the control cable and the tubular guidewire at the proximal end of the tubular guidewire, provides resistance to the control cable movement. The resistive force maintains the position of the control cable relative to the tubular guidewire during a procedure. Alternatively, the sheathless filter may be locked into position either by a torque device tightened over the tubular guidewire, by a clamp, or by an interference fit created by a projection on the control cable mating with the inner diameter surface of the tubular guidewire. At the distal end of the control cable, radial expansion of the sheathless filter is limited to maintain appropriate maximum allowable mesh spacing. A stop is crimped onto the control cable beyond the distal end of the tubular guidewire. The stop blocks control cable retraction into the tubular guidewire at the acceptable limit of deployment. Alternatively, the location at which the proximal end of the sheathless filter is joined to the tubular guidewire can be used to control the extent of deployment.

In one embodiment for coronary vascular procedures, the tubular guidewire preferably has an effective length of 180 cm. The outer diameter of the tubular guidewire would be 0.014 inch. Starting profile of the sheathless filter is 0.030 inch and fully expanded profile would be 0.3 inch. There is no deployment delay as with inflating a balloon. Deployment is immediate upon activation of the control cable. This embodiment in combination with an aspiration debris removal device is particularly adapted to provide distal embolization protection in debulking vascular interventional procedures, such as those involving a blocked saphenous vein coronary bypass graft. The aspiration debris removal device removes the majority of particles while the sheathless filter captures the remainder. Alternatively, the present invention may be configured and sized for use in peripheral vascular procedures or neurovascular procedures.

The advantage of the protective system or apparatus of the present invention is that it behaves like an ordinary guidewire yet does not require a bulky sheath for deployment or retrieval. Unlike balloon occlusive devices that block a vessel, the sheathless filter of the present invention allows for the continuous flow of blood, thus decreasing potential damage to downstream tissue. Unlike other filters, the sheathless filter of the present invention has a variable diameter based on the extent of deployment, which further results in a range of filtration capabilities. Moreover, the flexible nature of the filter mesh conforms to vessel shape and the “soft” multifilament polymer fibers create less damage. There are no complicated mechanical arrangements or valve systems internal or external to the protective system or apparatus.

### BRIEF DESCRIPTION OF THE DRAWINGS

FIG. 1 is a side view of the protective system or apparatus prior to expansion of the sheathless filter.

FIG. 2 is a side view of the protective system or apparatus after expansion of the sheathless filter.

FIG. 3 is a close-up side view of a portion of the protective system or apparatus featuring the sheathless filter prior to expansion.

FIG. 4 is a close-up side view of a portion of the protective system or apparatus featuring the sheathless filter partially expanded.

FIG. 5 is a close-up side view of a portion of the protective system or apparatus featuring the sheathless filter at full operational expansion.

FIG. 6 is a detail view of the braided wire framework and filter mesh of the sheathless filter.

FIG. 7 is a side view of the protective system or apparatus with a clamp attached to the control cable.

FIG. 8 is a side view of another embodiment of protective system or apparatus.

FIGS. 9A, 9B and 9C are detailed cross sectional views of the sheathless filter of the embodiment shown in FIG. 8.

FIG. 10 is a detailed side view of the flexible guidewire tip of the embodiment shown in FIG. 8.

FIG. 11 is a magnified view of a section of the filter mesh of one embodiment of the sheathless filter.

FIG. 12 is a detailed view of the proximal attachment of the sheathless filter to the tubular guidewire.

FIGS. 13 and 14 are cross sectional views of an interference fit used with one embodiment of the present invention.

### DETAILED DESCRIPTION OF THE PREFERRED EMBODIMENTS

The present invention is a protective system or apparatus for use in vascular procedures. The protective system or apparatus includes a tubular guidewire having a proximal end, a distal end, and a lumen; a control cable, having a proximal end and a distal end, disposed in the lumen of the tubular guidewire; and a sheathless filter distally coupled to the control cable and proximally coupled to the tubular guidewire. The sheathless filter expands in response to the displacement of the control cable relative to the tubular guidewire such that the sheathless filter presents at least a proximal exterior convex primary filter surface to the flow of blood in a blood vessel. In one embodiment, the sheathless filter has a distal interior concave surface which provides a secondary filter surface to the flow of blood within a blood vessel.

The protective system or apparatus is preferably provided with a mechanism or means for resisting displacement of the control cable relative to the tubular guidewire. In one embodiment, a short tube is disposed intermediate the tubular guidewire and the control cable at the proximal end of the tubular guidewire. The short tube is crimped to resist movement of the control cable. When the control cable is adjusted, it thus remains in place due to the resistance created by the short tube. In another embodiment, the control cable contains a stop so as to limit displacement. In a further embodiment, a clamping mechanism is used to selectively clamp the control cable to resist displacement. Alternatively, the control cable may be equipped with structure to provide an interference fit with the interior of the lumen of the tubular guidewire.

The sheathless filter is preferably comprised of wire elements that form a tubular braided wire framework over which other members are woven to create the filter mesh. In one embodiment, the other members are multifilament polymer fibers. In an alternate embodiment, the other members are nitinol wires of different diameters. The wire elements used for the braided wire framework are biocompatible and have material properties consistent with that needed to create a tubular braided structure. For example, nitinol wire elements could be used in this application. In an alternate embodiment, the multifilament polymer fibers that create the filter mesh could be woven into a fabric and then attached to the braided wire framework.

To allow deployment of the sheathless filter, the control cable is longer than the tubular guidewire and has a smaller diameter than the inner diameter of the tubular guidewire. Preferably, the outer diameter of the tubular guidewire is 0.018 inch or less. In addition, the proximal end of the tubular guidewire will be free of any mechanical connections and obstructions so as to enable the tubular guidewire to function as a conventional exchange guidewire while the sheathless filter is deployed.

The present invention provides a method of preventing plaque, thrombus or grumous material and debris from flowing downstream during vascular procedures. The method includes guiding a tubular guidewire into a blood vessel until a sheathless filter located at the distal end of the tubular guidewire is positioned distal to the region of the blood vessel to be treated. A control cable coaxially disposed within the tubular guidewire and affixed to the sheathless filter is displaced, thus expanding the sheathless filter to a deployed state which spans the diameter of the blood vessel. The tubular guidewire is clamped at the proximal end so as to prevent unwanted further displacement of the control cable during the vascular procedure. In a preferred embodiment, the tubular guidewire has an outer diameter of up to 0.018 inch and is made of nitinol or comparable material.

A catheter is introduced over the proximal end of the tubular guidewire and is advanced to the region of the blood vessel to be treated. A vascular procedure is then performed in the area using the catheter. The present invention may be incorporated with a vascular procedure such as an asymmetric water jet atherectomy wherein a jet directs a working fluid at a velocity sufficient to generate a stagnation pressure for removal of ablated deposit debris. The catheter is also used to remove material captured by the proximal exterior convex primary filter surface of the sheathless filter. The control cable is then released thus contracting the sheathless filter. The tubular guidewire with sheathless filter is then guided out of the blood vessel.

In the preferred method, the sheathless filter is comprised of a braided wire framework over which other members are co-braided to create a barrier to particles. The braided wire framework is attached to the distal end of the control cable and to the proximate end of the tubular guidewire. The braided wire framework and co-braided other members are selectively spaced so that particles are captured. In one embodiment, the co-braided other members are multifilament polymer fibers. In an alternate embodiment, the co-braided other members are nitinol wires of different diameters. Preferably, the co-braided other members and braided wire framework are spaced to capture particles of at least 250 microns and more preferably down to 100-150 microns. In the alternative, the multifilament polymer fibers are woven into a fabric and then attached to the braided wire framework. Before deployment, the sheathless filter has a closed position in which the braided wire framework and multifilament polymer fibers or other members are disposed generally parallel to the control cable and tubular guidewire so that the sheathless filter can be inserted into a blood vessel.

The present invention is also a system or apparatus for filtering emboli from the blood of a patient generally coincident to a vascular procedure. The system or apparatus includes blood vessel lumen opening means, debris filtering means, and emboli evacuation means. To open the lumen of a blood vessel, a catheter is used which has a distal end having one or more orifices from which a working fluid, such as saline under high pressure, is directed in the form of a fluid jet at a deposit within the blood vessel. The fluid jet impacts the deposit longitudinally so as not to damage the blood vessel. The impact dislodges the deposit and creates a plurality of debris particles.

The blood vessel lumen opening means further includes a tubular member containing a hypotube. Preferably, the tubular member is used as an evacuation lumen. The hypotube further includes one or more high velocity fluid jets directed to strike a portion of the tubular member. The high velocity fluid jets create a localized low pressure region which draws the debris particles to the fluid jets and subsequently down the exhaust lumen.

The debris filtering means includes the sheathless filter which is advanced distally of the deposit by the tubular guidewire. The sheathless filter is comprised of a braided wire framework over which a plurality of strands are co-braided. The braided wire framework is radially deployed. The braided wire framework is fixed at its proximal end to the tubular guidewire and the braided wire framework is fixed at its distal end to the control cable. In a non-deployed state, the individual wire elements of the braided wire framework lie generally parallel to the control cable and the tubular guidewire.

The sheathless filter can be selectively deployed so as to radially expand to span the diameter of the blood vessel. At a lower deployment limit, no fluid is able to pass through the sheathless filter. In a first embodiment, the lower deployment limit for the sheathless filter would be 3 mm. Likewise, the sheathless filter has an upper deployment limit based on the unoccupied distance between any two of the strands. This unoccupied distance is defined as a pore size. In a first embodiment, the maximum pore size is 0.010 inch in each direction of the opening so that the sheathless filter would be able to capture particles of 250 microns or greater. Alternatively, the maximum pore size is 0.005 inch so that the sheathless filter is able to capture particles of 100-150 microns or greater. In an alternate embodiment, the multifilament fibers are woven into a fabric prior to attaching them to the braided wire framework.

In one embodiment, the debris capturing means includes two filtering surfaces. First, a proximal exterior convex filter surface of the sheathless filter blocks the passage of particles immediately downstream of the vascular procedure. A second debris capturing means includes an interior concave filter surface at the distal end of the sheathless filter.

The outer diameter of the sheathless filter is smaller than the inner diameter of the blood vessel prior to deployment. In a first embodiment, it is envisioned that the sheathless filter will have an outer diameter of no more than 0.038 inch prior to deployment.

In operation, it is envisioned that emboli evacuation would include removing the displaced emboli from the proximal exterior convex filter surface of the sheathless filter through the use of the evacuation lumen. The evacuation lumen is attached to a vacuum pump which provides suction at the distal end. In addition, the evacuation lumen may be driven by the fluid jets which create a stagnation pressure upon striking the mouth of the evacuation lumen. Emboli evacuation is further accomplished due to the interior concave filter surface at the distal end of the sheathless filter catching emboli that are not trapped by the proximal convex filter surface. The sheathless filter is contracted and removed from the body upon completion of the procedure. The trapped emboli are maintained within the sheathless filter body during the removal procedure.

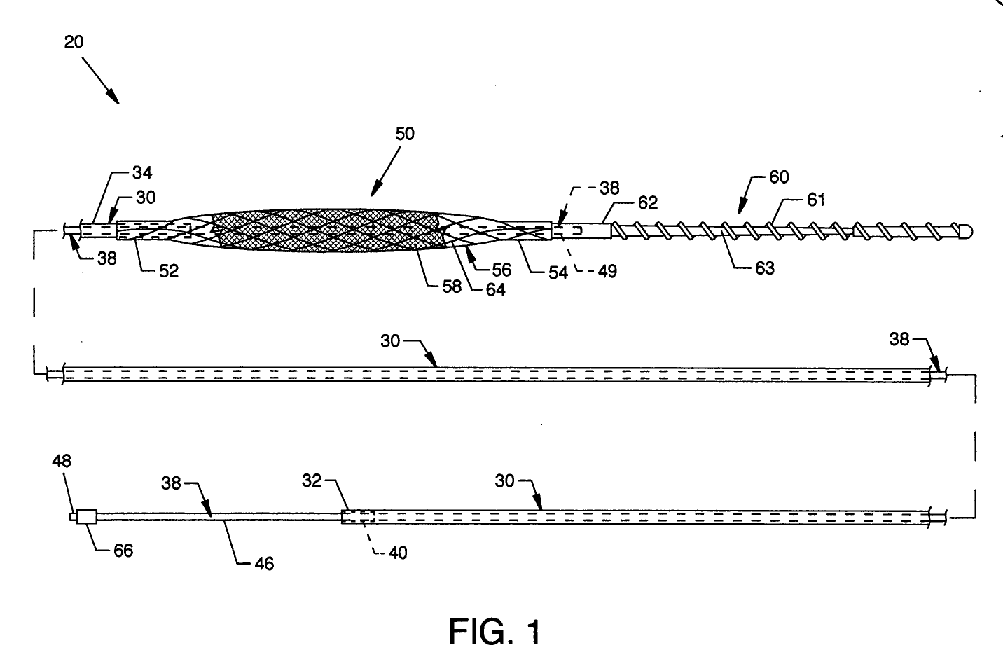

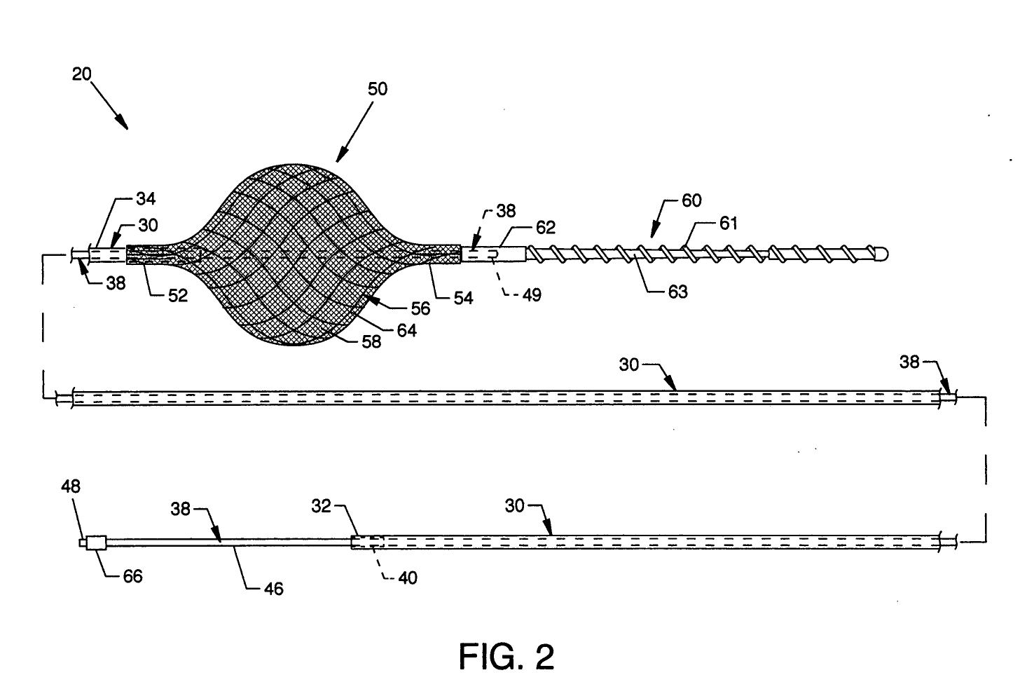

Referring now to FIGS. 1-2, the overall structure and operation of a protective system or apparatus 20 in accordance with the present invention will be described. The protective system or apparatus 20 includes a tubular guidewire 30, a sheathless filter 50, and a control cable 38.

The tubular guidewire 30 includes a proximal end 32 and a distal end 34. As used in the present invention, the terms proximal and distal will be used with reference to an operator, such that the distal end 34 of the tubular guidewire 30, for example, is the portion first inserted into a blood vessel, and the proximal end 32 is the portion which remains exterior to the patient and is therefore closer to the operator.

The tubular guidewire 30 receives the control cable 38 and an optional short tube 40.

The control cable 38 is a wire having a proximal end 48 and a distal end 49 slidably disposed within the lumen of the tubular guidewire 30. The control cable 38 extends proximally and distally beyond the respective proximal and distal ends 32 and 34 of the tubular guidewire 30. The total length of control cable 38 is longer than the length of the tubular guidewire 30 to provide for the sheathless filter 50 at the distal end 34 and a gripping region 46 at the proximal end 32. Exact lengths for the respective elements are determined by the required path to reach the occlusive site within the patient.

In a preferred embodiment, a flexible guidewire tip 60 is positioned in a sleeve 62 which is attached to the control cable 38 at the distal end 49 of control cable 38 to assist in deployment of the protective system or apparatus 20 through a blood vessel. In one embodiment, a stop 66 is crimped onto proximal end 48 of control cable 38 to prevent the control cable 38 from completely sliding into the lumen of tubular guidewire 30.

In one embodiment, travel of the control cable 38 is restricted by a short tube 40 disposed within the lumen of tubular guidewire 30 at the proximal end 32. The short tube 40 has an inner diameter slightly larger than the diameter of control cable 38 and an outer diameter slightly smaller than the inner diameter of tubular guidewire 30. The short tube 40 increases the resistive effect on the control cable 38 so as to maintain position relative to tubular guidewire 30. The short tube 40 ordinarily is less than one inch in length. The tubular guidewire 30 is crimpable relative to the short tube 40.

Alternatively, the short tube 40 could be eliminated and position then maintained by crimping the proximal end 32 of the tubular guidewire 30 to the control cable 38, by using a torque device, by using a clamp, by the interaction of a projection on the control cable 38 with the interior diameter surface of the tubular guidewire 30, or simply by maintaining relative position manually. Although the diameter of the control cable 38 could be of any size consistent with effective use of the tubular guidewire 30, it will be understood that the larger diameter creates a resistive effect on the tubular guidewire 30, or short tube 40, so as to maintain position relative to the tubular guidewire 30 when force is removed.

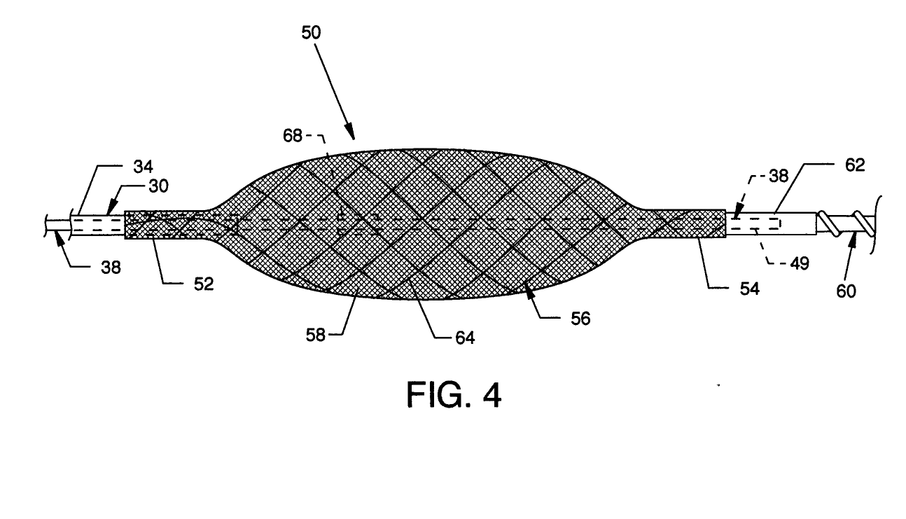

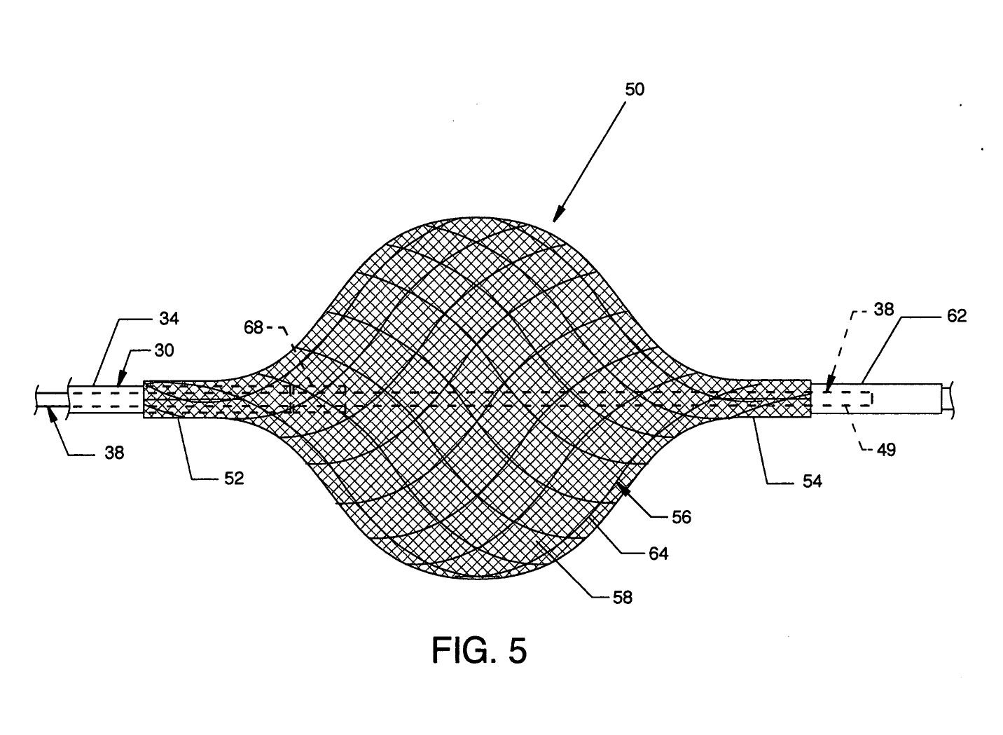

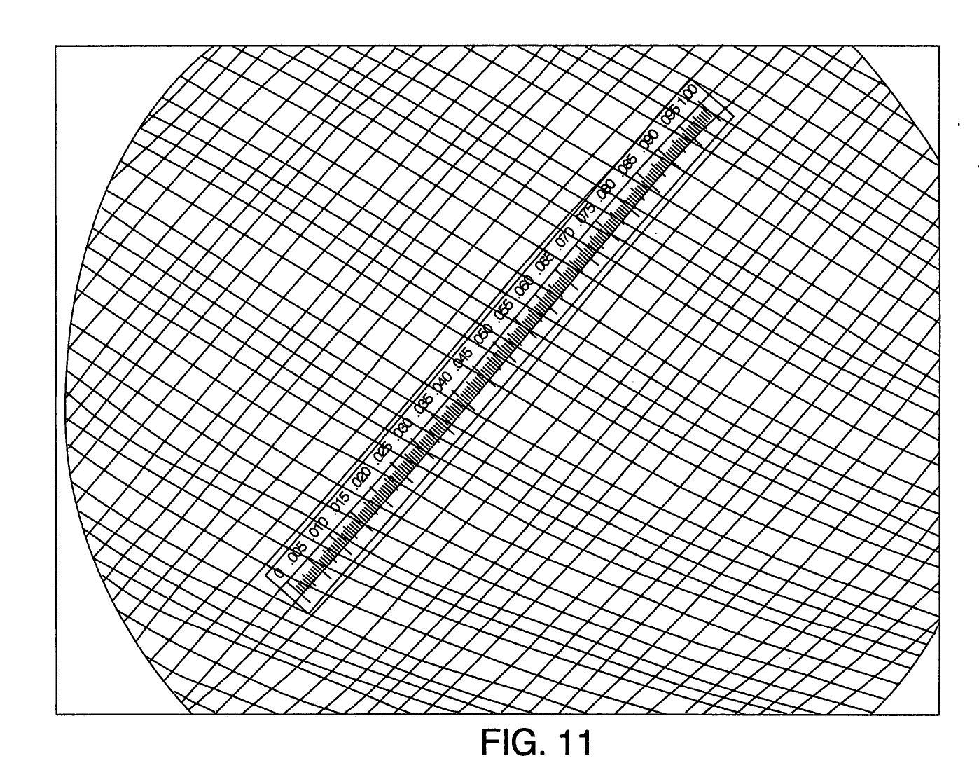

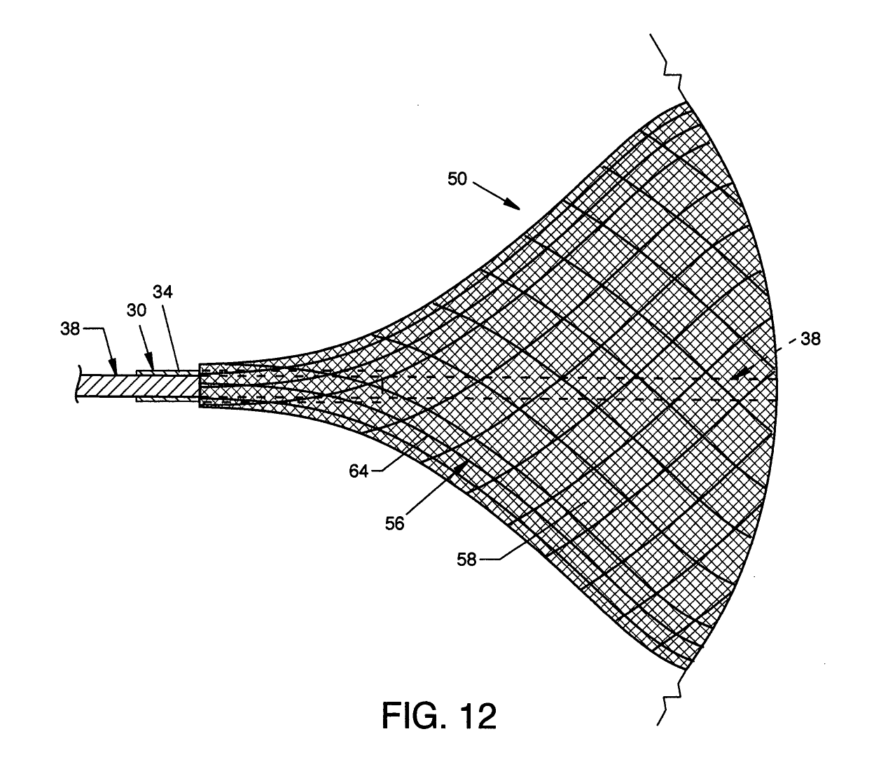

A sheathless filter 50 having a proximal end 52 and a distal end 54 is located at the distal end 34 of tubular guidewire 30. The sheathless filter 50 is preferably comprised of a braided wire framework 56 over which a plurality of multifilament polymer fibers are braided to form a filter mesh 58. Alternatively, the braided wire framework 56 may support a filter mesh 58 of other fibers or wires, such as nitinol wires, as shown in FIG. 11. The proximal end 52 of the sheathless filter 50 is laser-welded to the distal end 34 of tubular guidewire 30 as shown in FIG. 12, for example, while the distal end 54 of the sheathless filter 50 is laser-welded to the distal end 49 of control cable 38. In one embodiment, a control cable stop 68 (see FIGS. 4 and 5) is disposed on control cable 38, between the proximal end 52 and distal end 54 of the sheathless filter 50, so as to limit the travel of control cable 38. Exact location of the stop 68 is determined by the filter spacing created upon radial expansion of the braided wire framework 56 as compared to the particle size to be filtered.

The individual wire elements 64 of braided wire framework 56 are disposed parallel to tubular guidewire 30 and control cable 38 at the points of attachment so as to present a minimal crossing profile. Individual polymer fibers are co-braided about the braided wire framework 56 to increase cross-section coverage without the stiffness associated with the wire elements 64. Alternatively, other members comprised of smaller diameter strands or wires that exhibit more flexibility than the wire elements 64 associated with the braided wire framework 56 may be used. In one embodiment, the sheathless filter 50 may be coated with a hemocompatible compound to minimize shear activation of platelets.

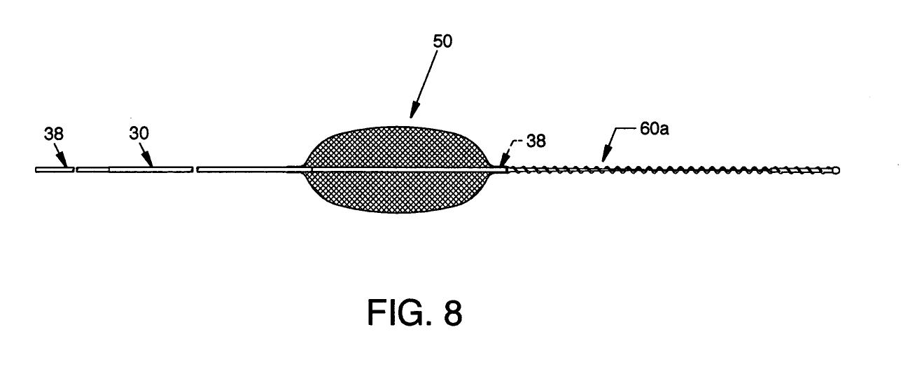

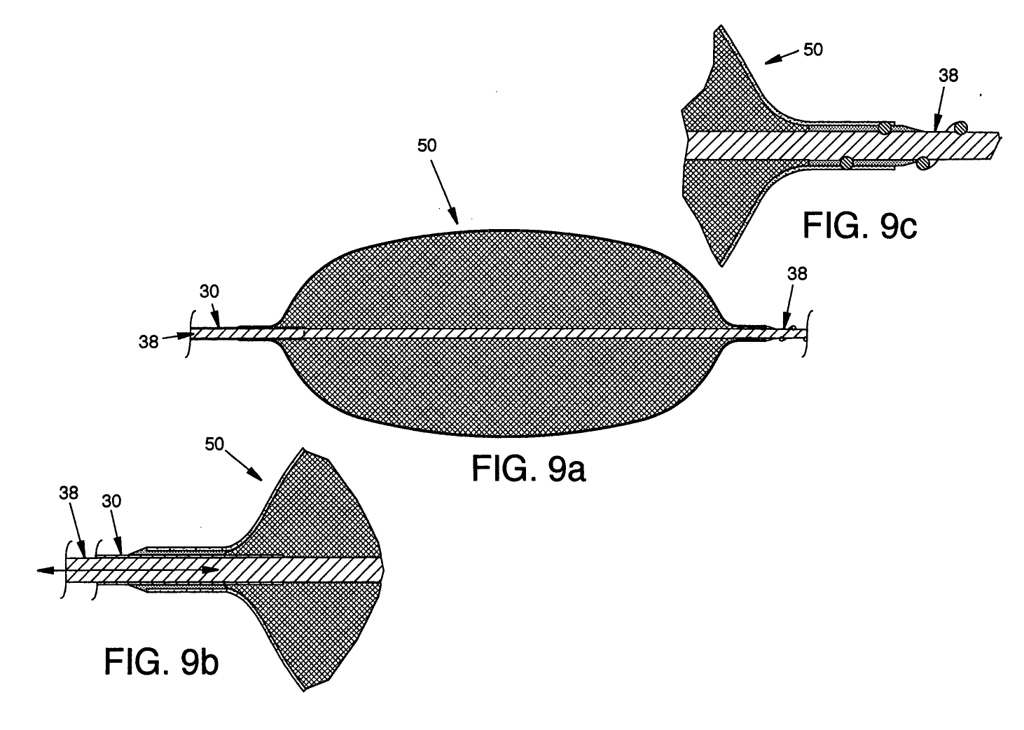

During interventional procedures involving carotid arteries and saphenous vein bypass grafts, embolic particles may be liberated causing adverse complications if preventive means are not in place. In a preferred embodiment, as illustrated in FIGS. 1-6, the protective system or apparatus 20 provides embolic protection. In one embodiment, the tubular guidewire 30 is formed of a nitinol tube having an outer diameter of 0.014 inch, an inner diameter of 0.010 inch, and a length of 180 cm. In an alternate embodiment as shown in FIGS. 8-10, the tubular guidewire 30 is formed of a braided polyimide tube having an outer diameter of 0.015 inch, an inner diameter of 0.011 inch, and a length of 180 cm, such as available from MedSource Technologies, Trenton, Ga. In one embodiment, the control cable 38 is formed of a nitinol wire having a diameter of 0.008 inch and a length of 190 cm. In an alternate embodiment, the control cable 38 is formed of a Teflon® coated stainless steel wire having a diameter of 0.0095 inch. The control cable 38 is disposed coaxially with the tubular guidewire 30. Although the length of the tubular guidewire 30 could be any length, it will be understood that it will be shorter than the length of control cable 38. In a first embodiment, the control cable 38 will be at a minimum of 10 cm longer so as to provide for the attachment of the sheathless filter 50 at the distal end 34 and a gripping region 46 at the proximal end 32. In one embodiment, short tube 40 is also preferably made of nitinol with a length of 0.5 inch.

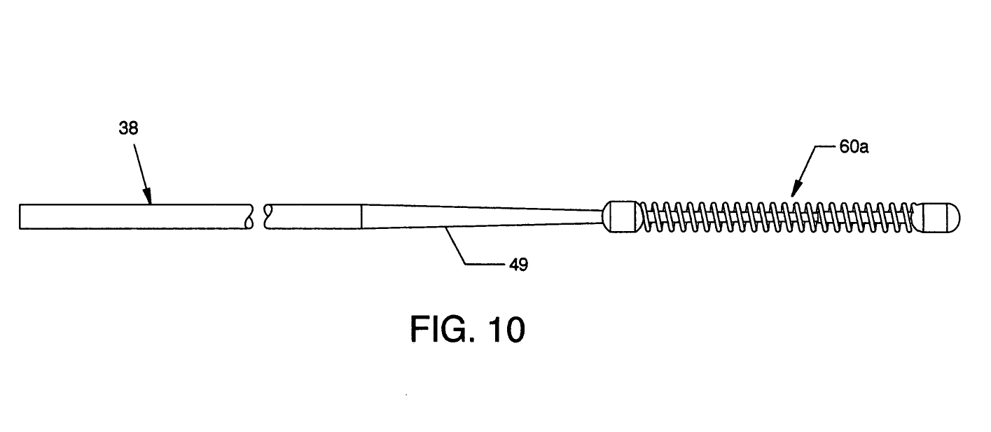

As shown in FIG. 1, the flexible guidewire tip 60 is disposed at the distal end 49 of the control cable 38. The flexible guidewire tip 60 is preferably a platinum coil 61 with a stainless steel core 63 having a maximum diameter of 0.018 inch and a length of 1.0 inch. Attachment of flexible guidewire tip 60 is accomplished in one embodiment by a stainless steel sleeve 62 that is laser-welded to control cable 38. A crimp is applied to the sleeve 62 to hold the flexible guidewire tip 60 in place. FIG. 10 shows an alternate embodiment flexible guidewire tip 60a wherein the core thereof is fabricated as part of the control cable 38.

In one embodiment, at the proximal end 48 of control cable 38, a stop 66 is attached. In this embodiment, stop 66 is 0.25 inch long with a diameter of 0.014 inch, which is equal to the diameter of tubular guidewire 30. The stop 66 is crimped onto control cable 38 and serves to keep the proximal end 48 of the control cable 38 from entering into the tubular guidewire 30.

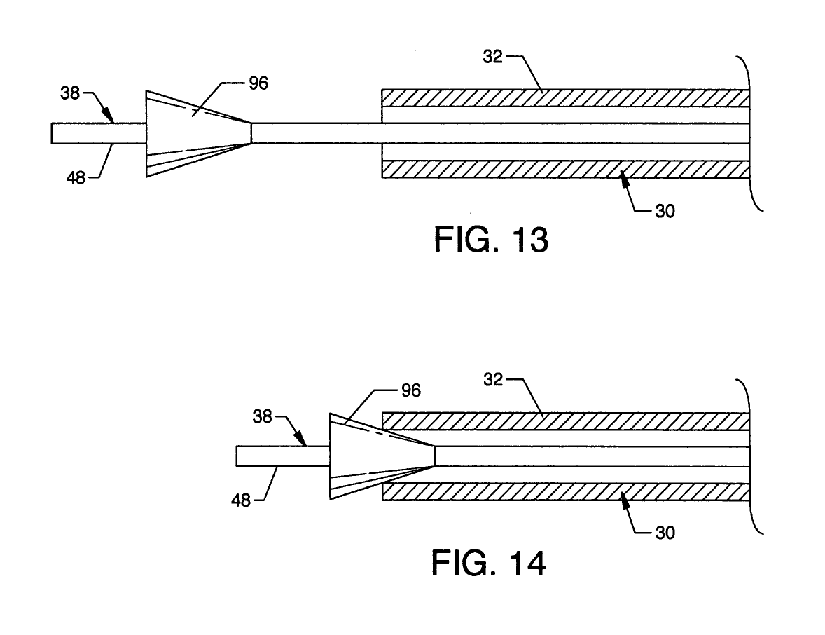

Another embodiment as shown in FIGS. 13 and 14 features an interference fit between the proximal end 48 of control cable 38 and the proximal end 32 of tubular guidewire 30 that effectively locks the sheathless filter 50 into a minimum diameter or undeployed state during insertion. This feature is particularly useful to ensure that the sheathless filter 50 remains in as unobtrusive a state as possible during passage through lesions or tortuous areas of the blood vessel undergoing a vascular procedure. The interference fit is created by a projection 96 on the control cable 38 which frictionally interfaces with the proximal opening of the lumen of the tubular guidewire 30 to secure the relative position between the two. In this embodiment, the projection 96 is a frustoconical-shaped member that extends beyond the outer diameter of the control cable 38. Numerous other shapes and configurations such as a lipped configuration or a ratchet arrangement could also be used.

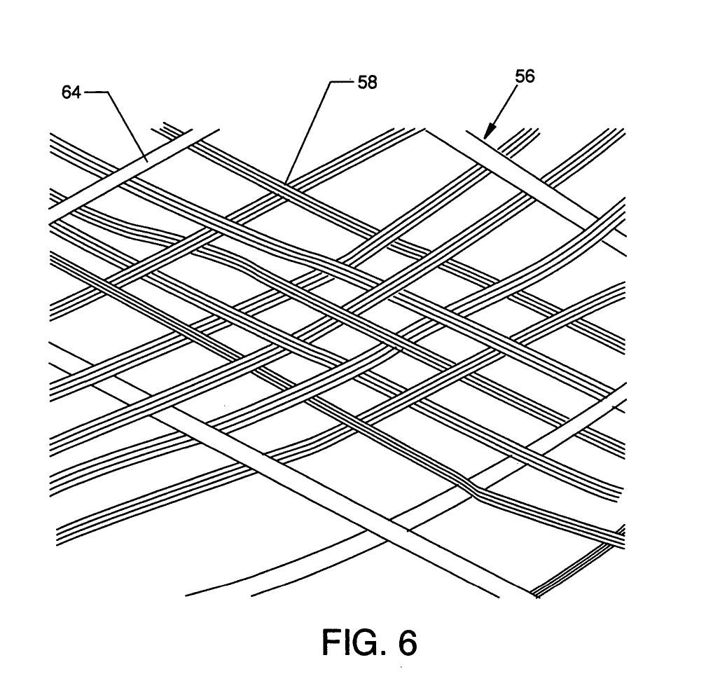

Sheathless filter 50 is comprised of a plurality of wire elements 64, which form a braided wire framework 56 to support a plurality of polymer fibers or strands formed into a filter mesh 58, as illustrated in FIG. 6. In a first embodiment, the polymer fibers or strands are co-braided around the braided wire framework 56. The wire elements 64 are made of nitinol, a super-elastic nickel titanium alloy, which is the preferred material because it is easy to braid and biocompatible. A plurality of laser-welds are applied at the proximal end 52 and distal end 54 to hold the ends of the wire elements 64 in position and prevent fraying. At least two welds are performed on each wire element 64 at each end so as to hold each wire element 64 as small as possible, thus presenting a minimal profile. Alternatively, adhesive bonds or mechanical interconnections may be used in place of or in addition to welding to secure the sheathless filter 50. In an alternate embodiment as shown in FIGS. 11 and 12, the strands forming the filter mesh 58 are also comprised of nitinol wire having a smaller diameter (e.g., 0.008 inch) than the nitinol wire elements 64 (e.g., 0.012 inch).

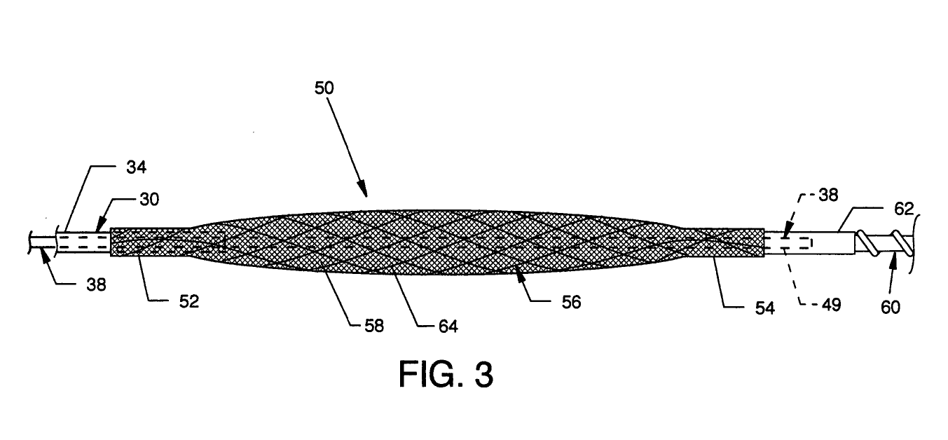

It is expected that the radial expansion of the sheathless filter 50 will have an upper and lower limit based on blood flow requirements at the lower limit and the ability to stop particles of an expected size at the upper limit. In a first embodiment, the lower limit of expansion would provide filtration in vessels as narrow as 3 mm. In order to filter particles of 250 microns or larger, the maximum allowable mesh gap would be 0.01 inch which corresponds to a maximum deployment diameter of 0.3 inch. In the closed position, as depicted in FIG. 3, the maximum diameter of the non-deployed sheathless filter 50 is 0.038 inch.

In another embodiment as shown in FIGS. 8-12, the sheathless filter 50 is designed to filter particles down to a size of between 100-150 microns. The inter-mesh spacings required for such a filtration effect range between 0.004 inch and 0.008 inch, as can be seen in FIG. 11, for example. In this embodiment, the expansion size of the sheathless filter 50 in a deployed state is selected among a plurality of sizes (e.g., 2-4 mm diameter vessels, 4-6 mm diameter vessels, 6-8 mm diameter vessels) to control the filtration effect of a given sized sheathless filter 50 by providing a known range of diameters in the deployed state for which the inter-mesh spacings necessary to achieve the desired filtration effect can then be chosen. In tests with the sheathless filter 50 of the present invention deployed within a 6.2 mm acrylic tube, polymer particles of known size were introduced into a fluid flow simulating blood to determine the effectiveness of the sheathless filter 50. When particles of a size of 200 microns were used in this test, 100% of the particles were trapped by the convex primary filter surface of the sheathless filter 50. When the particle size was reduced to 157 microns, 50% of the particles were trapped by the convex primary filter surface, 40% were trapped by the concave secondary filter surface and approximately 10% of the particles flowed through the sheathless filter 50. It will be seen that the sizes of the inter-spacing pores may be adjusted if protection for smaller size particles is desired to improve the effectiveness of the sheathless filter 50 for particles at those smaller sizes while potentially reducing the flow of blood due to the use of the smaller size pores.

To limit radial expansion of sheathless filter 50, in one embodiment stop 68 (see FIGS. 4 and 5) is coaxially disposed on control cable 38 between the proximal and distal ends 52 and 54 of sheathless filter 50. Stop 68 is a stainless steel tube crimped onto control cable 38 with an outer diameter of 0.012 inch, which stops the control cable 38 from traveling into the lumen of tubular guidewire 30.

The fibers, strands or wires of the filter mesh 58 and the wire elements 64 of the braided wire framework 56 lie generally parallel to control cable 38 when inserted into a blood vessel. As control cable 38 is proximally extended, distal end 54 is drawn toward stationary proximal end 52. Initial displacement, as depicted in FIG. 4, for example, creates a narrow tube as the sheathless filter 50 expands radially in an elastic manner to form a thin tube. As the control cable 38 is further displaced, the sheathless filter 50 continues its radial expansion, as depicted in FIG. 5, until stop 68 reaches distal end 34 or filling of the blood vessel takes place.

It will be understood that the weave of sheathless filter 50 may be varied in a number of ways including: changing the number of filaments per strand of the multifilament polymer fibers; changing the diameter of the polymer filaments; changing the number of nitinol wire elements which form the braided wire framework 56; changing the diameter of the nitinol wires and/or wire elements; and changing the design of the tubular weave. A further advantage to this design is the “softness” created by the polymer fibers as they interact with the blood vessel. Varying the nitinol wire elements has a direct effect on the stiffness of the sheathless filter 50 and the “softness.” However, the number of nitinol wire elements must be sufficient to adequately constrain the multifilament polymer fibers. Clearly, the options described above may be used to tighten or relax the weave of the sheathless filter 50. Furthermore, the options may be combined to achieve comparable results.

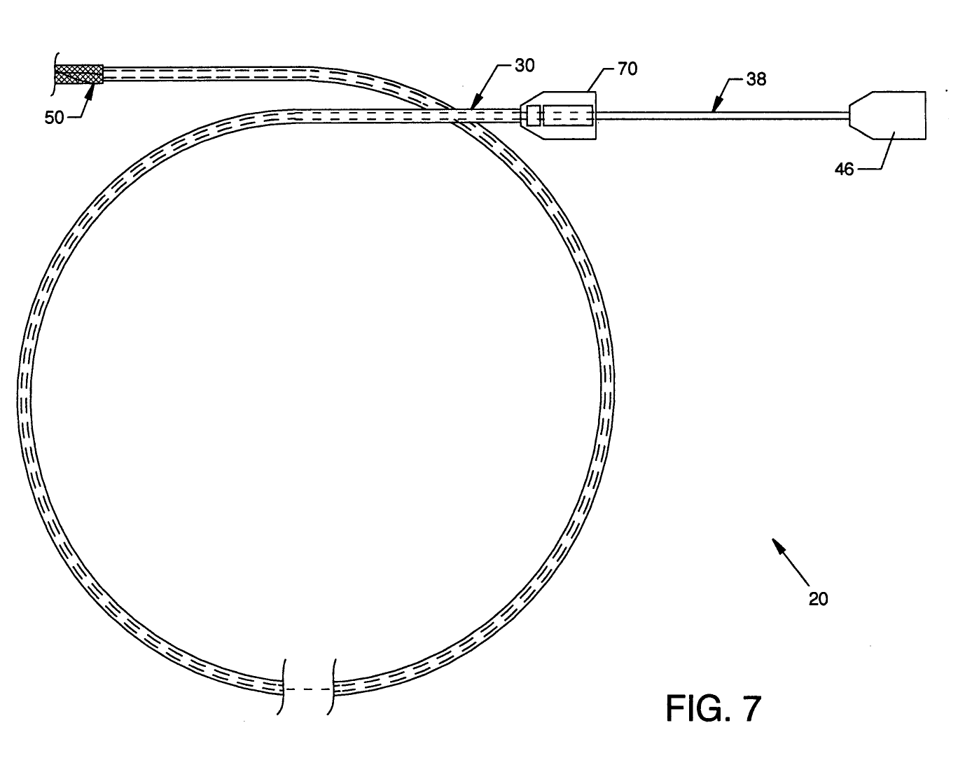

In practice, medical personnel gain access to the blood vessel lumen through which the protective system or apparatus 20 will travel. The protective system or apparatus 20 is removed from biocompatible packaging. Flexible guidewire tip 60 is inserted into the blood vessel lumen and is manipulated to a point beyond the vessel occlusion. The control cable 38 is drawn proximally from the tubular guidewire 30 so as to radially deploy the sheathless filter 50 within the blood vessel lumen. A rapid exchange device, such as a stent catheter or thrombectomy device, is then deployed on the tubular guidewire 30 with the sheathless filter 50 in a deployed state. As illustrated in FIG. 7, a clamp 70 is then applied to the control cable 38 to maintain the deployed position of the sheathless filter 50 until completion of the procedure.

In a preferred embodiment of the present invention, the protective system or apparatus 20 is utilized in an atherectomy or thrombectomy procedure of the type described in U.S. Pat. Nos. 5,370,609 or 5,496,267, the disclosure of each of which is hereby incorporated by reference. In each of these embodiments, the protective system or apparatus 20 is introduced into the patient, the sheathless filter 50 is radially deployed, and then the atherectomy or thrombectomy catheter arrangement is slid over the proximal end 32 of the tubular guidewire 30 and advanced until it is proximate and proximal to the location of the sheathless filter 50. Unlike other occlusive methods, the time period of the procedure is not constrained by concern over blockage of the blood vessel. The radial expansion of sheathless filter 50 allows for the continual flow of blood through the spacing between individual strands of the filter mesh 58. Thus, sheathless filter 50 is preferable where ischemia is intolerable or further blood cessation would be irreparably damaging.

Preferably, an evacuation of any debris dislodged in the therapy is accomplished by the evacuation lumen incorporated within the catheter assembly of the above-referenced patents. However, should debris escape the evacuation lumen, the proximal exterior convex filter surface of the sheathless filter 50 provides the primary filtering surface for trapping this detritus. The distal interior concave filter surface of the sheathless filter 50 provides a secondary filtering surface. Additionally, a sponge could be compressed to fit within the collapsed sheathless filter 50. Upon deployment, the sponge would provide a third level of filtering. After completion of the procedure, the sheathless filter 50 is returned to an undeployed state and the tubular guidewire 30 and sheathless filter 50 are retracted.

The present invention may be embodied in other specific forms without departing from the essential attributes thereof; therefore, the illustrated embodiments should be considered in all respects as illustrative and not restrictive, reference being made to the appended claims rather than to the foregoing description to indicate the scope of the invention.

Various modifications can be made to the present invention without departing from the apparent scope thereof.

### Protective System or Apparatus Including Guidewire Having Deployable Sheathless Filter and Method Utilizing Same

### Parts List

20 protective system or apparatus
30 tubular guidewire
32 proximal end
34 distal end
38 control cable
40 short tube
44 distal end
46 gripping region
48 proximal end
49 distal end
50 sheathless filter
52 proximal end
54 distal end
56 braided wire framework
58 filter mesh
60 flexible guidewire tip
60a flexible guidewire tip
61 platinum coil
62 sleeve
63 stainless steel core
64 wire elements
66 stop
68 stop
70 clamp
96 projection

1. Apparatus for use in vascular procedures comprising:
a. a tubular guidewire having a proximal end, a distal ends, and a lumen;
b. a control cable having a proximal end and a distal end disposed in the lumen of the tubular guidewire; and,
c. a sheathless filter distally coupled to the control cable and proximally coupled to the tubular guidewire, the sheathless filter being radially expandable in response to displacement of the control cable relative to the tubular guidewire such that the sheathless filter presents at least a convex primary filter surface to a flow of blood within a blood vessel when introduced thereinto and expanded.

2. The apparatus of claim 1, further including means for resisting displacement of the control cable relative to the tubular guidewire.

3. The apparatus of claim 2, wherein the means for resisting displacement comprises a short tube disposed intermediate the tubular guidewire and the control cable, the short tube being crimpable to selectively resist movement of the control cable and maintain a position of the control cable relative to the tubular guidewire.

4. The apparatus of claim 2, wherein the means for resisting displacement comprises a clamping mechanism to selectively clamp the control cable to resist movement of the control cable and to maintain a position of the control cable relative to the tubular guidewire.

5. The apparatus of claim 2, wherein the means for resisting displacement comprises a stop that limits displacement of the control cable relative to the tubular guidewire, the stop being disposed between the distal and proximal ends of the sheathless filter.

6. The apparatus of claim 1, wherein the sheathless filter comprises:
a. a tubular braided wire framework; and,
b. multifilament polymer fibers woven onto the tubular braided wire framework.

7. The apparatus of claim 6, wherein the tubular braided wire framework is constructed of biocompatible wire.

8. The apparatus of claim 7, wherein the biocompatible wire is nitinol wire.

9. The apparatus of claim 6, wherein the multifilament polymer fibers are woven into a fabric that is then attached to the tubular braided wire framework.

10. The apparatus of claim 6, wherein a distal end of the tubular braided wire framework is operably attached to the control cable and a proximal end of the tubular braided wire framework is operably attached to the tubular guidewire.

11. The apparatus of claim 6, wherein the tubular braided wire framework and the multifilament polymer fibers are spaced with respect to each other so as to define a maximum pore size of 0.010 inch that will effectively capture particles greater than 250 microns in diameter.

12. The apparatus of claim 1, wherein the sheathless filter includes means for visibly identifying the sheathless filter under fluoroscopy.

13. The apparatus of claim 1, wherein the sheathless filter includes a distal interior face presenting a concave secondary filter surface to the flow of blood within the blood vessel.

14. The apparatus of claim 1, wherein the proximal end of the tubular guidewire is free of mechanical connections and obstructions so as to enable the tubular guidewire to function as a conventional exchange guidewire while the sheathless filter is deployed.

15. The apparatus of claim 1, wherein the sheathless filter has an outer diameter of a maximum of 0.038 inch.

16. The apparatus of claim 1, wherein the sheathless filter is formed of resilient flexible members interlaced to form a tubular net, the tubular net having an undeployed state in which the flexible members lie generally parallel to a longitudinal axis of the control cable and tubular guidewire and having a plurality of selectively deployable states in which the flexible members are radially expanded from the longitudinal axis of the control cable and tubular guidewire to a diameter coincident with a diameter of the blood vessel.

17. The apparatus of claim 16, wherein the plurality of selectively deployable states include a state in which the flexible members are radially expanded and effectively abut each other such that blood is unable to pass through the sheathless filter.

18. The apparatus of claim 16, wherein the plurality of selectively deployable states include a state in which the flexible members define a pore size between adjacent members that is a maximum of 0.010 inch so as to filter particles greater than 250 microns.

19. A method of protecting against plaque, thrombus or grumous material flowing downstream during a vascular procedure, the method comprising:
a. guiding a tubular guidewire into a blood vessel and positioning a sheathless filter proximate a distal end of the tubular guidewire distal to a region of the blood vessel to be treated;
b. displacing a control cable coaxially disposed with the tubular guidewire to cause expansion of the sheathless filter to span a diameter of the blood vessel and present at least a convex surface to a flow of blood within the blood vessel;
c. selectively securing the control cable relative to the tubular guidewire to maintain a position of the sheathless filter during the vascular procedure;
d. performing the vascular procedure;
e. introducing a thrombectomy catheter over a proximal end of the tubular guidewire and advancing the thrombectomy catheter to the region of the blood vessel to be treated;
f. removing plaque, thrombus or grumous material captured by the sheathless filter during the vascular procedure via the thrombectomy catheter;
g. releasing the control cable relative to the tubular guidewire and causing the sheathless filter to contract; and,
h. withdrawing the tubular guidewire from the blood vessel.

20. The method of claim 19, wherein the vascular procedure comprises an asymmetric water jet atherectomy.

21. The method of claim 19, wherein the vascular procedure comprises an asymmetric water jet thrombectomy.

22. The method of claim 19, wherein the step of removing material involves utilizing a water jet that directs a working fluid at a velocity sufficient to generate a stagnation pressure large enough for removal of the material.

23. The method of claim 19, wherein the step of removing material involves utilizing aspiration to remove the material.

24. A system for filtering and removing plaque, thrombus or grumous material coincident with a vascular procedure comprising:
a. a guidewire having a sheathless filter positioned proximate a distal end of the guidewire, the sheathless filter being selectively deployable such that the sheathless filter presents at least a convex filter surface to a flow of blood within a blood vessel when introduced into the blood vessel and deployed prior to the vascular procedure;
b. an evacuation catheter having an evacuation lumen to be tracked over the guidewire and at least one evacuation opening proximate a distal end of the evacuation lumen; and,
c. means for removing plaque, thrombus or grumous material captured by the sheathless filter during the vascular procedure via the evacuation lumen of the evacuation catheter prior to the sheathless filter being selectively undeployed and the guidewire removed from the vessel.

25. The system of claim 24, further comprising:
a. a therapeutic catheter having a fluid lumen and trackable over the guidewire as part of the vascular procedure, the fluid lumen including at least one orifice proximate a distal end and opening to a side of the therapeutic catheter; and
b. means for supplying a working fluid under high pressure to the fluid lumen of the therapeutic catheter such that the working fluid is directed from the at least one orifice as a fluid jet stream longitudinally impacting on a deposit in the blood vessel to erode the deposit and generate free floating plaque, thrombus or grumous material in the blood vessel proximal to the sheathless filter.

26. The system of claim 25, wherein the therapeutic catheter and the evacuation catheter comprise a single catheter.

27. The system of claim 26, wherein the therapeutic catheter includes a plurality of orifices and the corresponding plurality of fluid jet streams create a localized low pressure region that draws plaque, thrombus or grumous material into the evacuation lumen.

28. The system of claim 24, wherein the guidewire has a proximal end, a distal end, and a lumen and further comprises a control cable having a proximal end and a distal end disposed in the lumen of the guidewire, wherein the sheathless filter is distally coupled to the control cable and proximally coupled to the guidewire.

29. The system of claim 28, further including means for resisting displacement of the control cable relative to the guidewire proximate the proximal end of the guidewire.

30. The system of claim 29, wherein the means for resisting displacement comprises a short tube disposed intermediate the guidewire and the control cable, the short tube being crimpable to selectively resist movement of the control cable and maintain a position of the control cable relative to the guidewire.

31. The system of claim 29, wherein the means for resisting displacement comprises a clamping mechanism to selectively clamp the control cable along the guidewire to resist movement of the control cable and maintain a position of the control cable relative to the guidewire.

32. The system of claim 29, wherein the means for resisting displacement comprises a stop that limits displacement of the control cable relative to the guidewire, the stop being disposed between the distal and proximal ends of the sheathless filter.

33. The system of claim 23, wherein the sheathless filter comprises:
a. a tubular braided wire framework; and,
b. multifilament polymer fibers woven onto the tubular braided wire framework.

34. The system of claim 33, wherein the tubular braided wire framework is constructed of biocompatible wire.

35. The system of claim 34, wherein the biocompatible wire is nitinol wire.

36. The system of claim 33, wherein the multifilament polymer fibers are woven into a fabric that is then attached to the tubular braided wire framework.

37. The system of claim 33, wherein a distal end of the tubular braided wire framework is operably attached to the control cable and a proximal end of the tubular braided wire framework is operably attached to the guidewire.

38. The system of claim 33, wherein the tubular braided wire framework and the multifilament polymer fibers are spaced with respect to each other so as to define a maximum pore size of 0.010 inch that will effectively capture particles greater than 250 microns in diameter.

39. The system of claim 24, wherein the sheathless filter includes means for visibly identifying the sheathless filter under fluoroscopy.

40. The system of claim 24, wherein the sheathless filter includes a distal interior face presenting a concave secondary filter surface to the flow of blood within the blood vessel.

41. The system of claim 24, wherein the proximal end of the guidewire is free of mechanical connections and obstructions so as to enable the guidewire to function as a conventional exchange guidewire while the sheathless filter is deployed.

42. The system of claim 24, wherein the sheathless filter has an outer diameter of a maximum of 0.038 inch.

43. The system of claim 24, wherein the sheathless filter is formed of resilient flexible members interlaced to form a tubular net, the tubular net having an undeployed state in which the flexible members lie generally parallel to a longitudinal axis of the control cable and guidewire and having a plurality of selectively deployable states in which the flexible members are radially expanded from the longitudinal axis of the control cable and guidewire to a diameter coincident with a diameter of the blood vessel.

44. The system of claim 43, wherein the plurality of selectively deployable states include a state in which the flexible members are radially expanded and effectively abut each other such that blood is unable to pass through the sheathless filter.

45. The system of claim 43, wherein the plurality of selectively deployable states include a state in which the flexible members define a pore size between adjacent members that is a maximum of 0.010 inch so as to filter particles greater than 250 microns.
